(19) United States
(12) Patent Application Publication
(10) Pub. No.: US 2026/0263543 A1
(43) Pub. Date: Sep. 10, 2026

(54) COMPOSITIONS AND METHODS TO INCREASE PRODUCTION OF GAMMA-AMINOBUTYRIC ACID

(71) Applicant: Verb Biotics, Boston, MA (US)

(72) Inventors: Todd Beckman, Boston, MA (US); Noah Zimmerman, Chapel Hill, NC (US); Camille Delebecque, Portsmouth, NH (US)

(73) Assignee: Verb Biotics, Boston, MA (US)

(21) Appl. No.: 19/658,073

(22) Filed: Apr. 24, 2026

Related U.S. Application Data
(63) Continuation of application No. PCT/IB2024/060546, filed on Oct. 25, 2024.
(60) Provisional application No. 63/593,827, filed on Oct. 27, 2023.
(60) Provisional application No. 63/602,766, filed on Nov. 27, 2023.

(51) Int. Cl. A61K 35/747; A61K 31/197; A61K 31/198; A61K 31/4415; A61K 31/675; A61K 35/00; A61P 25/22

(52) U.S. Cl. CPC A61K 35/747; A61K 31/197; A61K 31/198; A61K 31/4415; A61K 31/675; A61P 25/22; A61K 2035/115

(57) ABSTRACT

Disclosed herein are compositions and methods for increasing GABA levels in a subject. GABA levels can be increased by the conversion of glutamate to GABA using the compositions and methods disclosed herein, or by administering supernatant which comprises GABA. The compositions and methods disclosed herein can be used to treat diseases or disorders caused by low levels of GABA. Accordingly, disclosed herein are compositions and methods of treating sleep disorders, mood disorders, and stress disorders.

### CROSS-REFERENCE TO RELATED APPLICATIONS

This application is a continuation application of International Application No. PCT/IB2024/060546, filed Oct. 25, 2024, which was published by the International Bureau in English on May 1, 2025, and which claims the benefit of priority to U.S. Provisional Application 63/593,827 filed on Oct. 27, 2023, and U.S. Provisional Application No. 63/602,766 filed on Nov. 27, 2023, the entireties of which are incorporated herein by reference.

### FIELD OF INVENTION

The present disclosure relates generally to microbial compositions for increasing the production of gamma-aminobutyric acid (GABA).

### BACKGROUND

Gamma-aminobutyric acid (GABA) plays a fundamental role in the central nervous system through its effects on GABA post-synaptic receptors. Dysregulation of GABA levels has profound and pervasive impacts on human health, including impacts on mood, memory, and stress, amongst others. Mechanistically, GABA acts on two receptor types, GABA_{A} and GABA_{B}. A majority of clinical efforts focused on drugs, including neurosteroids, that are GABAergic, i.e., affecting the GABA system. This is often through agonistic or antagonistic activity on the GABA receptors (Backstrom et al., 2022; Zhang et al., 2021). But these approaches do not address GABA levels directly.

The ability to produce GABA is not unique to mammals but is also found in plants and microorganisms across a range of genera (Sarasa et al., 2020). Certain bacterial species, including those found in the gut microbiome of humans, are capable of producing GABA. However, the gut microbiome is a complex, multifactorial system—and its impacts on the central nervous system remain poorly understood (Miri et al., 2023). There have been efforts to utilize microorganisms to impact metabolite levels, including GABA, in human subjects, but these efforts are limited by the microorganism's ability to convert glutamate to GABA in vivo.

Accordingly, there exists a need in the art for new bacterial strains and compositions that can effectively increase levels of GABA in a subject.

### SUMMARY OF INVENTION

Compositions and methods for increasing levels of gamma-aminobutyric acid (GABA) in a subject are provided. Accordingly, in one aspect disclosed herein is a bacterial strain composition comprising a bacterial cell of Lactiplantibacillus plantarum Lp815 or an active variant of Lactiplantibacillus plantarum Lp815 or any combination thereof. In some embodiments, the Lactiplantibacillus plantarum Lp815 cell, active variant, or any combination thereof is present at about 10⁶ CFU/gram to about 10¹⁰ CFU/gram or at about 10⁶ CFU/mL to about 10¹⁰ CFU/mL. In some embodiments, the bacterial strain composition further comprises glutamate, pyridoxal phosphate, and/or vitamin B6. In some embodiments, the composition increases the conversion of glutamate to GABA. In some embodiments, the composition increases the conversion of glutamate to GABA at a pH of about 5.5 to about 7.0. In some embodiments, the composition increases the conversion of glutamate to GABA at a pH of about 7. In some embodiments, the composition is stable at room temperature for at least about 24 months. In some embodiments, the composition is stable after exposure to 65° C. for 15 seconds. In some embodiments, the composition further comprises GABA.

In one aspect, disclosed herein is a composition comprising a supernatant of Lactiplantibacillus plantarum Lp815 or an active variant thereof. In some embodiments, the supernatant further comprises cell fragments of Lactiplantibacillus plantarum Lp815 or an active variant thereof. In some embodiments, the composition further comprises GABA. In some embodiments, the composition further comprises a bacterial cell of Lactiplantibacillus plantarum Lp815 or an active variant of Lactiplantibacillus plantarum Lp815. In some embodiments, the composition comprises a capsule, gel, paste, tablet, powder, or liquid.

In one aspect, disclosed herein is a pharmaceutical composition comprising a pharmaceutically acceptable carrier and the compositions of the instant disclosure.

In one aspect, disclosed herein is a method of increasing GABA levels in a subject, the method comprising administering an effective amount of a pharmaceutical composition disclosed herein to the subject, when compared to an appropriate control. In some embodiments, the subject is a human subject. In some embodiments, an amount of supplemental glutamate is administered to the subject, wherein the amount of supplemental glutamate is between about 1 μg to about 1 g. In some embodiments, an amount of supplemental pyridoxal phosphate is administered to the subject, wherein the amount of supplemental pyridoxal phosphate is between about 10 μg to about 2000 mg. In some embodiments, an amount of supplemental Vitamin B6 is administered to the subject, wherein the amount of supplemental Vitamin B6 is between about 10 μg to about 2000 mg.

In some embodiments, the supplemental glutamate is simultaneously administered with the pharmaceutical composition. In some embodiments, the glutamate and the pharmaceutical composition are combined into a formulated product. In some embodiments, the supplemental glutamate is sequentially administered with an effective amount of the pharmaceutical composition, wherein the glutamate is administered either before, during, or after administration of the pharmaceutical composition.

In some embodiments, the supplemental pyridoxal phosphate is simultaneously administered with the pharmaceutical composition. In some embodiments, the pyridoxal phosphate and pharmaceutical composition are combined into a formulated product. In some embodiments, the supplemental pyridoxal phosphate is sequentially administered with the pharmaceutical composition. In some embodiments, the supplemental pyridoxal phosphate is administered either before, during, or after administration of the pharmaceutical composition.

In some embodiments, the supplemental Vitamin B6 is simultaneously administered with the pharmaceutical composition. In some embodiments, the Vitamin B6 and pharmaceutical composition are combined into a formulated product. In some embodiments, the supplemental Vitamin B6 is sequentially administered with the pharmaceutical composition. In some embodiments, the Vitamin B6 is administered either before, during or after administration of the pharmaceutical composition.

In some embodiments, the pharmaceutical composition, supplemental glutamate, supplemental pyridoxal phosphate, and/or supplemental Vitamin B6 is administered orally.

In one aspect, disclosed herein is a method of treating stress disorders in a subject, the method comprising administering to the subject an effective amount of a composition comprising a bacterial cell of Lactiplantibacillus plantarum Lp815, an active variant of Lactiplantibacillus plantarum Lp815, or a combination thereof; and/or a supernatant of Lactiplantibacillus plantarum Lp815 or active variant thereof, and a pharmaceutically acceptable carrier.

In one aspect, disclosed herein is a method of treating mood disorders in a subject, the method comprising administering to the subject an effective amount of a composition comprising a bacterial cell of Lactiplantibacillus plantarum Lp815, an active variant of Lactiplantibacillus plantarum Lp815, or a combination thereof; and/or a supernatant of Lactiplantibacillus plantarum Lp815 or active variant thereof, and a pharmaceutically acceptable carrier.

In one aspect, disclosed herein is a method of treating sleep disorders in a subject, the method comprising administering to the subject an effective amount of a composition comprising a bacterial cell of Lactiplantibacillus plantarum Lp815, an active variant of Lactiplantibacillus plantarum Lp815, or a combination thereof; and/or a supernatant of Lactiplantibacillus plantarum Lp815 or active variant thereof, and a pharmaceutically acceptable carrier.

In some embodiments of the above methods, the composition further comprises glutamate, pyridoxal phosphate, and/or Vitamin B6. In some embodiments, an amount of supplemental glutamate is administered to the subject, wherein the amount of supplemental glutamate is between about 1 μg to about 1 g. In some embodiments, an amount of supplemental pyridoxal phosphate is administered to the subject, wherein the amount of supplemental pyridoxal phosphate is between about 10 μg to about 2000 mg. In some embodiments, an amount of supplemental Vitamin B6 is administered to the subject, wherein the amount of supplemental Vitamin B6 is between about 10 μg to about 2000 mg.

In some embodiments, the supplemental glutamate is simultaneously administered with the pharmaceutical composition. In some embodiments, the glutamate and the pharmaceutical composition are combined into a formulated product. In some embodiments, the supplemental glutamate is sequentially administered with an effective amount of the pharmaceutical composition, wherein the glutamate is administered either before, during, or after administration of the pharmaceutical composition.

In some embodiments, the supplemental pyridoxal phosphate is simultaneously administered with the pharmaceutical composition. In some embodiments, the pyridoxal phosphate and pharmaceutical composition are combined into a formulated product. In some embodiments, the supplemental pyridoxal phosphate is sequentially administered with the pharmaceutical composition. In some embodiments, the supplemental pyridoxal phosphate is administered either before, during, or after administration of the pharmaceutical composition.

In some embodiments, the supplemental Vitamin B6 is simultaneously administered with the pharmaceutical composition. In some embodiments, the Vitamin B6 and pharmaceutical composition are combined into a formulated product. In some embodiments, the supplemental Vitamin B6 is sequentially administered with the pharmaceutical composition. In some embodiments, the Vitamin B6 is administered either before, during or after administration of the pharmaceutical composition.

In some embodiments, the supernatant of Lactiplantibacillus plantarum Lp815 or active variant thereof comprises GABA. In some embodiments, the supernatant of Lactiplantibacillus plantarum Lp815 or active variant thereof further comprises cell fragments. In some embodiments, the methods disclosed herein increase levels of GABA in the subject. In some embodiments, the effective amount of the composition comprises about 10⁶ CFU/gram to about 10¹⁰ CFU/gram or at about 10⁶ CFU/mL to about 10¹⁰ CFU/mL of the Lactiplantibacillus plantarum Lp815 cell, active variant of Lactiplantibacillus plantarum Lp815, or any combination thereof. In some embodiments, the composition, supplemental glutamate, supplemental pyridoxal phosphate, and/or supplemental Vitamin B6 is administered orally to the subject. In some embodiments, the composition, supplemental glutamate, supplemental pyridoxal phosphate, and/or supplemental Vitamin B6 is administered topically to the subject.

In some embodiments, the effective amount of the composition is between about 1×10⁹ to 1×10¹⁰ cfu. In some embodiments, the composition is administered once daily. In some embodiments, the composition results in a reduction in a GAD-7 score.

In some embodiments of the above, the Lactiplantibacillus plantarum Lp815 strain is NRRL Deposit No. B-68326.

### BRIEF DESCRIPTION OF THE FIGURES

FIG. 1A-1B demonstrate the increase in GABA (FIG. 1A) and 5-HIAA (FIG. 1B) observed in subjects that received the L. plantarum Lp815 strain.

FIG. 2 depicts the reduction in GAD-7 scores at baseline, 2 weeks, 4 weeks, and 6 weeks in subjects that received the L. plantarum Lp815 strain.

### DETAILED DESCRIPTION

Provided herein is a strain of Lactiplantibacillus plantarum (i.e., L. plantarum Lp815) which is capable of efficiently converting glutamate to gamma-aminobuytric acid (GABA). GABA is the primary inhibitory neurotransmitter in mammals and plays a critical role in modulating activity in the central nervous system through its effects on GABA_{A} and GABA_{B} post-synaptic receptors (Koh et al., 2023). Low GABA levels are known to be associated with stress, depression, insomnia, and mood disorders.

GABA is typically synthesized from glutamate by decarboxylation via the glutamic acid decarboxylase (GAD) enzyme:

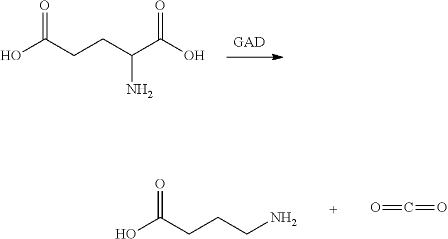

Numerous microorganisms have been identified that are naturally capable of producing GABA de novo, including Lactobacillus brevis, Lactiplantibacillus plantarum, and Bacteroides fragilis, amongst others. The efficiency of GABA conversion for a given species can be modulated by temperature, pH, nutrient availability, and other factors. While the synthesis of GABA via glutamate decarboxylation is the canonical pathway, other pathways for GABA synthesis are known in the art. Certain Pseudomonas sp. and Escherichia sp. can convert putrescine to GABA via a putrescine transaminase (see Kurihara et al., (2005), J. Biol. Chem. Vol 280 6:4602-4608 and Getino et al., (2023), Genes 14 (10): 1897). In yeast, GABA is catabolized first to succinic semialdehyde and then succinate, which feeds into the TCA cycle (see Cao et al., (2013) Yeast, 30 (4): 129-144).

Accordingly, disclosed herein is a strain of Lactiplantibacillus plantarum (e.g., Lactiplantibacillus plantarum Lp815) capable of producing unexpectedly high levels of GABA. The L. plantarum Lp815 strain disclosed herein can be used to treat diseases and disorders caused by low GABA levels.

### Definitions

In order that the disclosure may be more readily understood, certain terms are first defined. In addition, it should be noted that whenever a value or range of values of a parameter are recited, it is intended that values and ranges intermediate to the recited values are also part of this disclosure. It is also to be noted that as used herein, and in the claims, the singular forms “a” and “the” include plural references unless the context clearly indicates otherwise.

As used herein, reference to the bacterial species “Lactiplantibacillus plantarum”, “Lactobacillus plantarum”, and “L. plantarum” are considered interchangeable.

As used herein, the term “mood disorders” refers to mental health disorders that are characterized by depression, bipolar disorders, and/or anxiety disorders. Mood disorders can refer to, but is not limited to, major depression disorder, dysthymia, bipolar disorder, substance-induced mood disorders, social anxiety disorder, panic disorder, phobias, generalized anxiety disorder, separation anxiety disorder, and the like.

As used herein, the term “sleep disorders” refers to, but is not limited to, insomnia, narcolepsy, excessive daytime sleepiness, sleep phase disorders, circadian disorders, rapid eye movement behavioral disorders, restless leg syndrome, and other sleep movement disorders.

As used herein, the term “stress-related disorders” refers to disorders that develop as a result of stress. “Stress” is the result of a feeling or thought, either conscious or unconscious, that derives from traumatic events or external pressures. Stress can be acute, episodic, or chronic. Stress-related disorders are well-characterized in the art, and include Reactive Attachment Disorder, Disinhibited Social Engagement Disorder, Post-traumatic Stress Disorder, Acute Stress Disorder, and Adjustment Disorder. A subject may suffer from one or more stress-related disorders.

As used herein, the term “is stable” refers to the ability for the bacterial strain disclosed herein to remain active (i.e., convert glutamate to GABA) after exposure to an environmental condition or conditions. For example, the bacterial strain disclosed herein is stable if it retains the ability to convert glutamate to GABA following exposure to high temperatures (e.g., pasteurization conditions), prolonged exposure to room temperature conditions, and/or exposure to neutral pH conditions. Stable bacterial strains can have less than 100% of the activity of the strain prior to high temperature or storage conditions. In some embodiments, the stable bacterial strain has 50-100% of the activity of the strain prior to high temperature or storage conditions. In some embodiments, the stable bacterial strain has 50-60%, 60-70%, 70-80%, 80-90%, or 90-100% of the activity of the strain prior to high temperature or storage conditions.

As used herein, the term “supernatant” refers to the aqueous solution separated from the cells, spores, and/or forespores (or active variants thereof) of the bacterial strain compositions disclosed herein. In some embodiments, the supernatant comprises GABA. In some embodiments, the supernatant comprises inactive cell fragment of L. plantarum Lp815.

As used herein, the term “isolated” encompasses a bacterium spore, or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the components with which they were initially associated.

As used herein, a substance is “pure” if it is substantially free of other components. The terms “purify”, “purifying”, and “purified” refers to a bacterium, spore, or other material that has been separated from at least some of the components with which it was associated either when initially produced or generated (e.g., whether in nature or in an experimental setting), or during any time after its initial production. A bacterium or spore or a bacterial population or a spore population may be considered purified if it is isolated at or after production, such as from a material or environment containing the bacterium or bacterial population or spore, and a purified bacterium or bacterial population or spore may contain other materials up to about 10%, about 20%, about 30%, about 40%, about 50%, about 60%, about 70%, about 80%, about 90%, or above about 90% and still be considered purified. In some embodiments, purified bacteria or spores and bacterial populations or spore populations are more than about 80%, about 85%, about 90%, about 91%, about 92%, about 93%, about 94%, about 95%, about 96%, about 97%, about 98%, about 99%, or more than about 99% pure. In specific embodiments, a culture of bacteria contains no other bacterial species in quantities to be detected by normal bacteriological techniques.

### Bacterial Strains

Disclosed herein is a bacterial strain that is capable of converting glutamate to GABA. This bacterial strain is Lactiplantibacillus plantarum Lp815 or an active variant thereof. Cell populations comprising L. plantarum Lp815 or an active variant thereof are provided, as well as populations of spores or forespores derived from the strain, or any preparation thereof. Thus, the compositions provided herein comprise as an active ingredient a cell population, spore, forespore, or combination thereof, of L. plantarum Lp815 or an active variant thereof. In another aspect, compositions provided herein are a supernatant produced by culturing L. plantarum. In some embodiments, the supernatant comprises inactive cell fragments of L. plantarum Lp815.

Lactiplantibacillus plantarum Lp815 was deposited with the Patent Depository of the National Center for Agricultural Utilization Research Agricultural Research Service, U.S. Department of Agriculture, 1815 North University Street, Peoria, Illinois 61604 U.S.A., on Oct. 26, 2023, and assigned NRRL No. B-68326. The deposit will be maintained under the terms of the Budapest Treaty on the International Recognition of the Deposit of Microorganisms for the Purposes of Patent Procedure. The deposit was made merely as a convenience for those of skill in the art, and is not an admission that a deposit is required under 35 U.S.C. § 112. The L. plantarum Lp815 strain disclosed herein was isolated with repeated rounds of non-natural selection pressure combined with advanced computational tools and high-throughput screening techniques specifically designed to select for high GABA production.

### Active Variants of a Bacterial Strain

Further provided are active variants of L. plantarum Lp815. Active variants of the strain provided herein includes, for example, any isolate, mutant, or otherwise modified bacterial strain of L. plantarum Lp815 that retains the ability to convert glutamate to GABA. An active variant includes a strain having all of the identifying characteristics of the recited strain.

The L. plantarum Lp815 strain disclosed herein has the ability to convert glutamate to GABA at close to neutral pH levels. In some embodiments, the L. plantarum Lp815 strain has the ability to convert glutamate to GABA at a pH range of about 5.5 to 7.0. In some embodiments, the L. plantarum Lp815 strain has the ability to convert glutamate to GABA at a pH of about 5.5 to 5.6, 5.6 to 5.7, 5.7 to 5.8, 5.8 to 5.9, 5.9 to 6.0, 6.0 to 6.1, 6.1 to 6.2, 6.2 to 6.3, 6.3 to 6.4, 6.4 to 6.5, 6.5 to 6.6, 6.6 to 6.7, 6.7 to 6.8, 6.8 to 6.9, 6.9 to 7.0. In some embodiments, the L. plantarum Lp815 strain has 100% of the ability to produce GABA in neutral pH (e.g., 6.5 to 7.0) as compared to acidic pH (e.g., 5.5 to 6.5). In some embodiments, the L. plantarum Lp815 strain has 50-100% of the ability to produce GABA in neutral pH as compared to acidic pH. In some embodiments, the L. plantarum Lp815 strain has 50-60%, 60-70%, 70-80%, 80-90%, or 90-100% of the ability to produce GABA in neutral pH as compared to acidic pH.

The L. plantarum Lp815 strain disclosed herein remains stable after exposure to high temperatures (i.e., thermal shock). In some embodiments, the L. plantarum Lp815 strain disclosed herein remains stable after exposure to temperatures of about between 45-50° C., 50-55° C., 55-60° C., 60-65° C., 65-70° C., 70-75° C., 75-80° C., 80-85° C., 85-90° C., 90-95° C., 95-100° C., 100-105° C., or 105-110° C. In some embodiments, the L. plantarum Lp815 strain disclosed herein remains stable for up to 24 months following thermal shock. In some embodiments, the L. plantarum strain disclosed herein remains stable after exposure to pasteurization conditions, with temperature ranges of between 65° C. to 91° C. In some embodiments, the pasteurization conditions are between 15 seconds and 45 seconds or more.

The L. plantarum Lp815 strain disclosed herein remains stable after exposure to acidic conditions. In some embodiments, the L. plantarum Lp815 strain disclosed herein remains stable after exposure to pH conditions ranging from 3.5 to 5.5. In some embodiments, the L. plantarum Lp815 strain disclosed herein remains stable after exposure to pH conditions ranging from 3.5 to 3.6, 3.6 to 3.7, 3.7 to 3.8, 3.8 to 3.9, 3.9 to 4.0, 4.0 to 4.1, 4.1 to 4.2, 4.2 to 4.3, 4.3 to 4.4, 4.4 to 4.5, 4.5 to 4.6, 4.6 to 4.7, 4.7 to 4.8, 4.8 to 4.9, 4.9 to 5.0, 5.0 to 5.1, 5.1 to 5.2, 5.2 to 5.3, 5.3 to 5.4, or 5.4 to 5.5. In some embodiments, the L. plantarum Lp815 strain remains stable after exposure to acidic pH conditions for up to 24 months.

Due to the aforementioned ability for the L. plantarum Lp815 strain disclosed herein to remain stable after exposure to high temperatures and/or acidic conditions, the L. plantarum Lp815 strain is suitable for use in a variety of food and beverage products. In some embodiments, the food and beverage products comprising the L. plantarum Lp815 strain or a supernatant thereof are exposed to high temperatures and/or acidic conditions. In some embodiments, the food and beverage products comprising the L. plantarum Lp815 strain or supernatant thereof are acidic.

By “modified bacterial strain” is intended a population wherein the strain has been modified (by selection and/or transformation) to have one or more additional traits of interest. Modified bacterial strains can be made through genetic engineering techniques and such engineered or recombinant bacterial strains grown to produce a modified population of bacterial strains. A recombinant bacterial strain can be produced by introducing polynucleotides into the bacterial host cell by transformation or by otherwise altering the native bacterial chromosome sequence, including but not limited to, gene editing approaches. Methods for transforming microorganisms are known in the art. See, generally, Hanahan, D. (1983) Studies on transformation of Escherichia coli with plasmids J. Mol. Biol. 166, 557-77; Seidman, C. E. (1994) In: Current Protocols in Molecular Biology, Ausubel, F. M. et al. eds., John Wiley and Sons, NY; Choi et al. (2006) J. Microbiol. Methods 64:391-397; Wang et al. 2010. J. Chem. Technol. Biotechnol. 85:775-778. Transformation may occur by natural uptake of naked DNA by competent cells from their environment in the laboratory. Alternatively, cells can be made competent by exposure to divalent cations under cold conditions, by electroporation, by exposure to polyethylene glycol, by treatment with fibrous nanoparticles, or other methods well known in the art.

Active variants of the various bacteria provided herein can be identified by employing, for example, methods that determine the sequence identity relatedness between the 16S ribosomal RNA, methods to identify groups of derived and functionally identical or nearly identical strains including multi-locus sequence typing (MLST), concatenated shared gene trees, whole genome alignment (WGA), average nucleotide identity, and MinHash (Mash) distance metric.

In one aspect, the active variants of L. plantarum Lp815 include strains that are closely related to the disclosed strain by employing the Bishop MLST method of organism classification as defined in Bishop et al. (2009) BMC Biology 7 (1) 1741-7007-7-3. Thus, in specific embodiments, an active variant of a bacterial strain disclosed herein includes a bacterial strain that falls within at least a 80%, 85%, 86%, 87%, 88%, 89%, 90%, 91%, 92%, 93%, 94%, 95%, 96%, 97%, 98%, 98.5%, 98.8%, 99%, 99.1%, 99.2%, 99.3%, 99.4%, 99.5%, 99.6%, 99.7%, 99.8%, or 99.9% sequence cut off employing the Bishop method of organism classification as set forth in Bishop et al. (2009) BMC Biology 7 (1) 1741-7007-7-3, which is hereby incorporated by reference in its entirety. Active variants of the bacteria identified by such methods will retain the ability to convert glutamate to GABA.

In some embodiments, the active variants of the bacterial strain disclosed herein include strains that are closely related to any of the disclosed strains on the basis of the Average Nucleotide Identify (ANI) method of organism classification. ANI (see, for example, Konstantinidis, K. T., et al., (2005) PNAS USA 102 (7): 2567-72; and Richter, M., et al., (2009) PNAS 106 (45): 19126-31) and variants (see, for example, Varghese, N.J., et al., Nucleic Acids Research (Jul. 6, 2015): gkv657) are based on summarizing the average nucleotides shared between the genomes of strains that align in WGAs. Thus, in specific embodiments, an active variant of bacterial strain L. plantarum Lp815 disclosed herein includes a bacterial strain that falls within at least a 90%, 95%, 96%, 97%, 97.5%, 98%, 98.5%, 98.8%, 99%, 99.5%, or 99.8% sequence cut off employing the ANI method of organism classification as set forth in Konstantinidis, K. T., et al., (2005) PNAS USA 102 (7): 2567-72, which is herein incorporated by reference in its entirety. Active variants of the bacteria identified by such methods will retain the ability to convert glutamate to GABA.

In particular embodiments, the active variants of the isolated bacterial strain disclosed herein include strain(s) that are closely related to L. plantarum Lp815 on the basis of 16S rRNA sequence identity. See Stackebrandt E, et al., “Report of the ad hoc committee for the re-evaluation of the species definition in bacteriology,” Int J Syst Evol Microbiol. 52 (3): 1043-7 (2002) regarding use of 16S rRNA sequence identity for determining relatedness in bacteria. In an embodiment, the active variant is at least 95% identical to L. plantarum Lp815 on the basis of 16S rRNA sequence identity, at least 96% identical to L. plantarum Lp815 on the basis of 16S rRNA sequence identity, at least 97% identical to L. plantarum Lp815 on the basis of 16S rRNA sequence identity, at least 98% identical to L. plantarum Lp815 on the basis of 16S rRNA sequence identity, at least 98.5% identical to L. plantarum Lp815 on the basis of 16S rRNA sequence identity, at least 99% identical to L. plantarum Lp815 on the basis of 16S rRNA sequence identity, at least 99.5% identical to L. plantarum Lp815 on the basis of 16S rRNA sequence identity, or at least 100% identical to L. plantarum Lp815 on the basis of 16S rRNA sequence identity. Active variants of the bacteria identified by such methods will retain the ability to convert glutamate to GABA.

The MinHash (Mash) distance metric is a comparison method that defines thresholds for hierarchical classification of microorganisms at high resolution and requires few parameters and steps (Ondov et al. (2016) Genome Biology 17:132). The Mash distance estimates the mutation rate between two sequences directly from their MinHash sketches (Ondov et al. (2016) Genome Biology 17:132). Mash distance strongly corresponds to Average Nucleotide Identity method (ANI) for hierarchical classification (See, Konstantinidis, K. T. et al. (2005) PNAS USA 102 (7): 2567-72, herein incorporated by reference in its entirety). That is, an ANI of 97% is approximately equal to a Mash distance of 0.03, such that values put forth as useful classification thresholds in the ANI literature can be directly applied with the Mash distance.

Active variants of the bacterial strain disclosed herein include strains that are closely related to L. plantarum Lp815 on the basis of the Mash distance between complete genome sequences. Thus, in specific embodiments, an active variant of a bacterial strain disclosed herein includes bacterial strains having a genome within a Mash distance of less than about 0.015 to the disclosed strains. In other embodiments, an active variant of a bacterial strain disclosed herein includes a distance metric of less than about 0.001, 0.0025, 0.005, 0.010, 0.015, 0.020, 0.025, or 0.030. A genome, as it relates to the Mash distance includes both bacterial chromosomal DNA and bacterial plasmid DNA. In other embodiments, the active variant of a bacterial strain has a genome that is above a Mash distance threshold to the disclosed strains that is greater than dissimilarity caused by technical variance. In further instances, the active variant of a bacterial strain has a genome that is above a Mash distance threshold to the disclosed strains that is greater than dissimilarity caused by technical variance and has a Mash distance of less than about 0.015. In other instances, the active variant of a bacterial strain has a genome that is above a Mash distance threshold to the disclosed strains that is greater than dissimilarity caused by technical variance and has a Mash distance of less than about 0.001, 0.0025, 0.005, 0.010, 0.015, 0.020, 0.025, or 0.030.

As used herein, “above technical variation” means above the Mash distance between two strains caused by errors in the genome assemblies provided the genomes being compared were each DNA sequenced with at least 20× coverage with the Illumina HiSeq 2500 DNA sequencing technology and the genomes are at least 99% complete with evidence for contamination of less than 2%. While 20× coverage is an art recognized term, for clarity, an example of 20× coverage is as follows: for a genome size of 5 megabases (MB), 100 MB of DNA sequencing from the given genome is required to have 20× sequencing coverage on average at each position along the genome. There are many suitable collections of marker genes to use for genome completeness calculations including the sets found in Campbell et al. (2013) PNAS USA 110 (14): 5540-45, Dupont et al. (2012) ISMEJ 6:1625-1628, and the CheckM framework (Parks et al. (2015) Genome Research 25:1043-1055); each of these references is herein incorporated in their entirety. Contamination is defined as the percentage of typically single copy marker genes that are found in multiple copies in the given genome sequence (e.g. Parks et al. (2015) Genome Research 25:1043-1055); each of these references is herein incorporated in their entirety. Completeness and contamination are calculated using the same collection of marker genes. Unless otherwise stated, the set of collection markers employed in the completeness and contamination assay is those set forth in Campbell et al. (2013) PNAS USA 110 (14): 5540-45, herein incorporated by reference.

Exemplary steps to obtain a distance estimate between the genomes in question are as follows: (1) Genomes of sufficient quality for comparison must be produced. A genome of sufficient quality is defined as a genome assembly created with enough DNA sequence to amount to at least 20× genome coverage using Illumina HiSeq 2500 technology. The genome must be at least 99% complete with contamination of less than 2% to be compared to the claimed microbe's genome. (2) Genomes are to be compared using the Minhash workflow as demonstrated in Ondov et al. (2016) Genome Biology 17:132, herein incorporated by reference in its entirety. Unless otherwise stated, parameters employed are as follows: “sketch” size of 1000, and “k-mer length” of 21. (3) Confirm that the Mash distance between the two genomes is less than 0.001, 0.0025, 0.005, 0.010, 0.015, 0.020, 0.025, or 0.030. Using the parameters and methods stated above, a Mash distance of 0.015 between two genomes means the expected mutation rate is 0.015 mutations per homologous position. Active variants of the bacteria identified by such methods will retain the ability to convert glutamate to GABA.

### Methods of Cultivating Bacterial Strains

Populations or cultures of L. plantarum Lp815 can be produced by cultivation of the bacterial strain. Cultivation can be started by scaling-up a seed culture. This involves repeatedly and aseptically transferring the culture to a larger and larger volume to serve as the inoculum for the fermentation, which can be carried out in large stainless steel fermenters in medium containing proteins, carbohydrates, and minerals necessary for optimal growth of the strain. Non-limiting exemplary media is Tryptic Soy Broth (TSB) or Lysogeny Broth (LB). After the bacterial inoculum is added to the fermentation vessel, the temperature and agitation are controlled to allow maximum growth. Once the culture reaches a maximum population density, the culture is harvested by separating the cells from the fermentation medium (i.e., supernatant). This separation is commonly performed by centrifugation.

The concentration of the bacterial culture can be measured from any sample of fermentation broth or bacterial strain composition. A colony forming unit (CFU) is the viable cell count of a sample resulting from standard microbiological plating methods. The term is derived from the fact that a single cell when plated on appropriate medium will grow and become a viable colony in the agar medium. Since multiple cells may give rise to one visible colony, the term colony forming unit can be a more useful unit measurement than cell number.

The compositions disclosed herein can comprise an amount of bacterial strain L. plantarum Lp815, or an active variant thereof, or spores, forespores, or a combination of cells, spores, and/or forespores formed from L. plantarum Lp815. Such amount can comprise a concentration of L. plantarum Lp815 of at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, or about 10⁹ CFU/g to about 10¹⁰ CFU/g. In other embodiments, the concentration of L. plantarum or an active variant thereof comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g. Another such an amount can comprise a concentration of L. plantarum of at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 105 CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, or about 10⁹ CFU/ml to about 10¹⁰ CFU/ml. In other embodiments, the concentration of L. plantarum Lp815 or active variants thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

In some of the embodiments as described above, the Lactiplantibacillus plantarum Lp815 strain is NRRL Deposit No. B-68326.

### Formulations of Bacterial Strains

The bacterial strain of L. plantarum Lp815 provided herein or active variants thereof can be formulated as a paste (e.g., a cell paste), a powder, a capsule, a tablet, a granule, a cell pellet, dust, a slurry, aqueous or oil based liquid products, gel, and the like.

Common bacterial strain compositions, such as probiotic preparations, are liquid solutions and concentrates or lyophilized powders for resuspension, which can be enclosed in a capsule, vial, or pouch. Such formulations will comprise the bacterial strains provided herein or an active variant thereof, in addition to carriers and other agents. As used herein, the term “carrier” refers to an inert compound that is compatible with any other ingredients in the formulation and is not deleterious to the active compound (i.e., bacterial strains) or a subject that the formulation is administered thereto. Suitable carriers can be added to improve recovery, efficacy, or physical properties and/or to aid in packaging and administration. Such carriers may be added individually or in combination. Non-limiting examples of carriers include proteins, carbohydrates, fats, enzymes, vitamins, immune modulators, oligosaccharides, milk replacers, minerals, amino acids, coccidiostats, acid-based products, medicines (such as antibiotics), other probiotics, and/or prebiotics. Common carriers include cellulose, sugar, glucose, lactose, whey powder, or rice hulls.

The carrier(s) may comprise about 30% weight per weight, weight per volume, or volume per volume, of the final composition. In some embodiments, the carrier(s) may comprise about 40%, about 50%, about 60%, about 70%, about 80%, about 90%, about 91%, about 92%, about 93%, about 94%, about 95%, about 96%, about 97%, about 98%, about 98.5%, about 99.0%, about 99.5%, or about 99.9% weight per weight, weight per volume, or volume per volume of the final composition.

In some embodiments, the bacterial strain composition comprises a pharmaceutical composition wherein the bacterial strains provided herein are formulated as a pharmaceutical composition along with a pharmaceutically acceptable carrier. Such pharmaceutically acceptable carriers are known in the art and include an inert vehicle, adjuvants, preservatives etc., which are well known. In some embodiments, the pharmaceutically acceptable carrier comprises one that is not naturally-occurring (i.e., not found in nature). In particular embodiments, the pharmaceutically acceptable carrier is a naturally-occurring carrier that is not found with a bacterial strain of the invention in the native environment of the bacterial strain (i.e., a pharmaceutically acceptable carrier that is not naturally-occurring with a bacterial cell, spore, or forespore of a bacterial strain of the invention). Such pharmaceutical compositions can be prepared in accordance with known techniques. See, e.g., Remington, The Science and Practice of Pharmacy (21st ed. 2005).

In some embodiments, feed and/or food compositions can be prepared by combining a formulated bacterial strain of the invention with typical animal feed and/or food or drink ingredients. A formulated bacterial strain of the invention can be used for the preparation of animal feed or food products or beverages, and/or may be added to drinking and/or rearing water. In other embodiments, the compositions of the present invention are feed, food and/or drink additives that are added to a subject's feed, food, drinking water or beverage prior to ingestion.

As used herein, “animal feed” includes any animal feed blend known in the art, including rapeseed meal, cottonseed meal, soybean meal, cornmeal, barley, wheat, silage, and haylage.

The bacterial strain of L. plantarum Lp815 or active variants thereof can be formulated as a food composition such as a dietary supplement, a functional food, a medical food or a nutritional product as long as the required effect is achieved, i.e. conversion of glutamate to GABA. Said food compositions may be chosen from the group consisting of beverages, yogurts, juices, ice creams, breads, biscuits, crackers, cereals, health bars, spreads, and nutritional products. The food composition may further comprise a carrier material, wherein said carrier material is chosen from the group consisting of lactic acid fermented foods, fermented dairy products, resistant starch, dietary fibers, carbohydrates, proteins and glycosylated proteins.

In some embodiments, the bacterial strain composition disclosed herein is formulated as a liquid formulation or a solid formulation. When the bacterial strain composition (e.g., pharmaceutical composition) is a solid formulation, it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip, or a film. When the bacterial strain composition (e.g., pharmaceutical composition) is a liquid formulation, it may be formulated as an oral solution, a suspension, an emulsion or syrup. The composition may further comprise a carrier material independently selected from, but not limited to, the group consisting of vegetables, lactic acid fermented foods, fermented dairy products, resistant starch, dietary fibers, carbohydrates, proteins, and glycosylated proteins

Compositions disclosed herein may also include prebiotics, which may be combined or mixed with a formulated bacterial strain of the invention into a feed or food composition, into drinking water, or into a pharmaceutical composition. Prebiotics are food ingredients that are not readily digestible by enzymes endogenous to the gut (such as those expressed by the animal or those expressed by the resident gut microbiome) and that selectively stimulate the growth and activity of selected groups of intestinal microorganisms that confer beneficial effects upon their host. Typically, it is beneficial microorganism populations that benefit from the presence of prebiotic compounds. Prebiotics can consist of oligosaccharides and other small molecules that serve as metabolic substrates for growth of beneficial microbes. Common prebiotics include galacto-oligosaccharides, fructo-oligosaccharides, inulin, isomalto-oligosaccharies, gentio-oligosaccharides, lactilol, lactosucrose, lactulose, xylosucrose, glycosylsucrose, pyrodextrins, soybean oligosaccharides, guar gum, locust bean gum, arabinan, galactan, pectins, and pectic polysaccharides. While many diverse microbes inhabit the intestinal tract of a host organism, prebiotic compounds are only utilized by the beneficial microbes and lead to a selective enhancement of the beneficial microbe population. A formulation that includes both prebiotics and probiotics may be known as a “symbiotic”.

In certain embodiments, the compositions disclosed herein comprise glutamate. In some embodiments, the composition comprises glutamate at an amount of about 1 μg to about 1 g, including but not limited to about 1 μg to about 2 μg, about 300 μg to about 100 mg, about 350 μg to about 30 mg, about 1 μg, about 2 μg, about 5 μg, about 10 μg, about 20 μg, about 25 μg, about 50 μg, about 100 μg, about 200 μg, about 300 μg, about 400 μg, about 500 μg, about 600 μg, about 700 μg, about 800 μg, about 900 μg, about 1 mg, about 2 mg, about 5 mg, about 10 mg, about 20 mg, about 25 mg, about 50 mg, about 100 mg, about 200 mg, about 300 mg, about 400 mg, about 500 mg, about 600 mg, about 700 mg, about 800 mg, about 900 mg, and about 1 g.

In some embodiments, the compositions disclosed herein comprise pyridoxal phosphate (or pyridoxal 5′-phosphate, used interchangeably throughout the disclosure). In some embodiments, the composition comprises pyridoxal phosphate at a concentration of about 1 to about 200 uM, including but not limited to about 1 to about 10 uM, about 10 uM to about 20 uM, about 20 uM to about 30 uM, about 30 uM to about 40 uM, about 40 uM to about 50 uM, about 50 uM to about 60 uM, about 60 uM to about 70 uM, about 70 uM to about 80 uM, about 80 uM to about 90 uM, about 90 uM to about 100 uM, about 100 uM to about 110 uM, about 110 uM to about 120 uM, about 120 uM to about 130 uM, about 130 uM to about 140 uM, about 140 uM to about 150 uM, about 150 uM to about 160 uM, about 160 uM to about 170 uM, about 170 uM to about 180 uM, about 180 uM to about 190 uM, about 190 uM to about 200 uM. In some embodiments, the composition comprises pyridoxal phosphate at a concentration of about 200 uM. In some embodiments, the composition comprises pyridoxal phosphate at an amount of about 0.1 mg to about 2000 mg. In some embodiments, the composition comprises pyridoxal phosphate at an amount of about 0.1 to about 100 mg, about 100 mg to about 500 mg, about 500 mg to about 1000 mg, about 1000 mg to about 1500 mg, about 1500 mg to about 2000 mg. In some embodiments, the composition comprises pyridoxal phosphate at an amount of about 1000 mg, about 1500 mg, or about 2000 mg.

In some embodiments, the compositions disclosed herein comprise Vitamin B6. In some embodiments, the composition comprises Vitamin B6 at a concentration of about 1 to about 200 uM, including but not limited to about 1 to about 10 μM, about 10 μM to about 20 μM, about 20 uM to about 30 μM, about 30 μM to about 40 μM, about 40 μM to about 50 μM, about 50 μM to about 60 μM, about 60 μM to about 70 μM, about 70 μM to about 80 μM, about 80 μM to about 90 μM, about 90 μM to about 100 μM, about 100 μM to about 110 μM, about 110 μM to about 120 μM, about 120 μM to about 130 μM, about 130 μM to about 140 μM, about 140 μM to about 150 μM, about 150 μM to about 160 μM, about 160 μM to about 170 μM, about 170 μM to about 180 μM, about 180 μM to about 190 μM, about 190 μM to about 200 μM. In some embodiments, the composition comprises Vitamin B6 at a concentration of about 200 μM. In some embodiments, the composition comprises Vitamin B6 at an amount of about 0.1 to about 100 mg, about 100 mg to about 500 mg, about 500 mg to about 1000 mg, about 1000 mg to about 1500 mg, about 1500 mg to about 2000 mg. In some embodiments, the composition comprises Vitamin B6 at an amount of about 1000 mg, about 1500 mg, or about 2000 mg.

In some embodiments, the compositions disclosed herein comprise glutamate and pyridoxal phosphate. In some embodiments, the compositions disclosed herein comprise glutamate and Vitamin B6. In some embodiments, the compositions disclosed herein comprise glutamate, pyridoxal phosphate, and Vitamin B6.

In some embodiments, the composition comprises a concentration of the bacterial strain of at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 105 CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, or about 10⁹ CFU/g to about 10¹⁰ CFU/g. In other embodiments, the concentration of the bacterial strain provided herein or active variant thereof comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g.

In liquid compositions and formulations, the amount of bacterial strain or active variant thereof, disclosed herein can comprise a concentration of at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, or about 10⁹ CFU/ml to about 10¹⁰ CFU/ml. In other embodiments, the concentration of the bacterial strain provided herein or active variant thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

In some of the embodiments as described above, the Lactiplantibacillus plantarum Lp815 strain is NRRL Deposit No. B-68326.

### Methods of Administration

Methods are provided herein for increasing the levels of GABA (via the conversion of glutamate to GABA) with an effective amount of a composition comprising a bacterial strain, or active variant thereof, disclosed herein. The methods comprise administering an effective amount of a composition comprising bacterial strain L. plantarum Lp815 or active variants thereof. These methods can be performed in vitro, in vivo, or ex vivo. When performed in vivo, the method comprises administering to a subject an effective amount of a composition disclosed herein. In specific embodiments, a composition further comprises supplemental glutamate, Vitamin B6, and/or pyridoxal phosphate to increase the production of GABA when compared to an appropriate control (e.g., a sample prior to the addition of the composition).

As used herein, an “effective amount” refers to a quantity of a composition comprising a bacterial strain disclosed herein that increases the production of GABA from glutamate when compared to an appropriate control (e.g., a sample prior to the addition of the composition). In some embodiments, an effective amount of the composition increases the level of GABA in a subject when compared to an appropriate control, treats one or more mood disorders in a subject when compared to an appropriate control, treats one or more sleep disorders in a subject when compared to an appropriate control, and/or treats one or more anxiety disorders in a subject when compared to an appropriate control.

An effective amount of the bacterial strain composition (e.g., pharmaceutical composition) is determined based on the intended goal. The term “unit dose” refers to a physically discrete unit suitable for use in a subject or agricultural animal, each unit containing a predetermined quantity of the bacterial strain composition calculated to produce the desired response in association with its administration, i.e., the appropriate route and treatment regimen. The quantity to be administered, both according to number of treatments and unit dose, depends on the subject to be treated, the state of the subject, the environmental conditions of the subject, and the result desired. Precise amounts of the bacterial strain composition also depend on the judgment of the practitioner and can be unique to each individual.

In specific embodiments, the effective amount of a bacterial strain, or active variant thereof, disclosed herein is about 10⁴ CFU to about 10¹² CFU, about 10⁵ CFU to about 10¹² CFU, about 10⁶ CFU to about 10¹² CFU, about 10⁷ CFU to about 10¹² CFU, about 10⁸ CFU to about 10¹² CFU, about 10⁹ CFU to about 10¹² CFU, about 10¹⁰ CFU to about 10¹² CFU, about 10¹¹ CFU to about 10¹² CFU, about 10⁵ CFU to about 10¹¹ CFU, about 10⁵ CFU to about 10¹¹ CFU, about 10⁶ CFU to about 10¹¹ CFU, about 10⁷ CFU to about 10¹¹ CFU, about 10⁸ CFU to about 10¹¹ CFU, about 10⁹ CFU to about 10¹¹ CFU, about 10¹⁰ CFU to about 10¹¹ CFU, about 10⁵ CFU to about 10¹⁰ CFU, about 10⁶ CFU to about 10¹⁰ CFU, about 10⁷ CFU to about 10¹⁰ CFU, about 10⁸ CFU to about 10¹⁰ CFU, or about 10⁹ CFU to about 10¹⁰ CFU. In other embodiments, the effective amount is at least about 10⁴ CFU, at least about 10⁵ CFU, at least about 10⁶ CFU, at least about 10⁷ CFU, at least about 10⁸ CFU, at least about 10⁹ CFU, at least about 10¹⁰ CFU, at least about 10¹¹ CFU, or at least about 10¹² CFU.

In some embodiments, a bacterial strain or an active variant thereof disclosed herein is administered to a subject that is also administered (simultaneously or sequentially) a prebiotic. Prebiotics, such as those described herein, may be combined with a strain disclosed herein into a formulated product or the prebiotic(s) may be administered separately from (before, during, or after) a bacterial strain disclosed herein.

In some embodiments, a bacterial strain or an active variant thereof disclosed herein is administered to a subject that is also administered (simultaneously or sequentially) glutamate. Glutamate may be combined with a strain disclosed herein into a formulated product, or the glutamate can be administered separately from (before, during, or after) a bacterial strain disclosed herein.

In some embodiments, the supernatant of a bacterial strain or an active variant thereof disclosed herein is administered to a subject. In some embodiments, the supernatant is administered simultaneously or sequentially with one or more compositions disclosed herein. The supernatant may be combined with a strain disclosed herein into a formulated product or the supernatant may be administered separately from (before, during, or after) a bacterial strain disclosed herein.

In those embodiments wherein the prebiotic, glutamate, and/or supernatant is administered separately from a bacterial strain composition, the bacterial strain composition may be administered before, during, or after the prebiotic, glutamate, and/or supernatant. The bacterial strain and the additional component can be administered to a subject within minutes (e.g., 1, 2, 5, 10, 15, 30, 45 minutes), hours (e.g., 1, 2, 3, 4, 5, 6, 7, 8, 9, 10, 12, 15, 20 hours), or days (e.g., 1, 2, 3, 4, 5, 6, 7 days) of each other.

As used herein, the compositions can be used to increase the levels of GABA in any subject when compared to an appropriate control (e.g., a sample from the subject prior to administration of the composition). By “subject’ is intended animals. In specific embodiments, the subjects are mammals, e.g., primates or humans.

As used herein, an “increase in”, or “increasing” GABA levels comprises any statistically significant increase in the level of GABA when compared to an appropriate control (e.g., a corresponding sample not contacted with a composition). As used herein, a “decrease in” or “decreasing” glutamate levels comprises any statistically significant decrease in the level of glutamate when compared to an appropriate control. Such increases can include, for example, at least a 5%, 10%, 20%, 30%, 40%, 50% or more increase in GABA levels when compared to an appropriate control. Such increases can also include, for example, at least about a 1-15%, 10-25%, 20-35%, 30-45%, 40-55% or more increase in GABA levels when compared to an appropriate control. Such decreases can include, for example, a 5%, 105%, 20%, 305%, 40%, 50% or more decrease in glutamate levels when compared to an appropriate control. Such decreases can also include, for example, at least about a 1-15%, 10-25%, 20-35%, 30-45%, 40-55% decrease in glutamate levels when compared to an appropriate control. GABA and/or glutamate levels can be measured in a bacterial composition. In some embodiments, the levels of GABA and/or glutamate can be measured in a sample taken from a subject or measured directly in the subject. Without wishing to be bound by any particular theory, it is believed that the levels of GABA and glutamate are inversely proportional in a subject. That is, an increase in GABA levels will correspond with a decrease in glutamate levels.

In specific embodiments, an increase in GABA levels is measured in reference to the level of GABA in a proper control sample. As used herein, a proper control includes but is not limited to, the level of GABA in a corresponding sample that does not comprise a bacterial strain disclosed herein, the level of GABA from a subject that was not administered a bacterial strain composition comprising a bacterial strain disclosed herein, the level of GABA in a sample from the subject prior to administration of a bacterial strain composition comprising a bacterial strain disclosed herein, or the level of GABA in a standardized sample from a subject that was not administered a bacterial strain composition comprising a bacterial strain disclosed herein. One of skill in the art would be able to identify proper controls in order to measure an increase in the level of GABA.

Thus, the presently disclosed methods comprise increasing GABA levels when compared to an appropriate control by administering an effective amount of a composition comprising bacterial strain L. plantarum Lp815. An appropriate control could be, but is not limited to the level of GABA in a corresponding sample that does not comprise bacterial strain L. plantarum Lp815, the level of GABA in a corresponding sample from a subject that was not administered bacterial strain L. plantarum Lp815, the level of GABA in a sample from the subject prior to administration of the bacterial strain composition comprising bacterial strain L. plantarum Lp815, and/or the level of GABA in a standardized sample from a subject that was not administered a composition comprising bacterial strain L. plantarum Lp815.

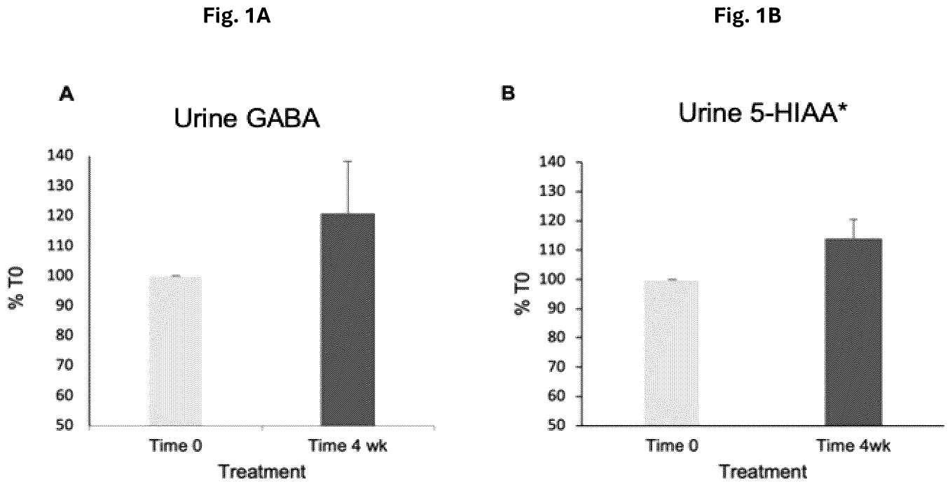

In some embodiments, the L. plantarum Lp815 strain increases the amount of GABA in a subject by at least 10%. In some embodiments, the L. plantarum Lp815 strain increases the amount of GABA in a subject by at least 20% (see FIG. 1A-1B).

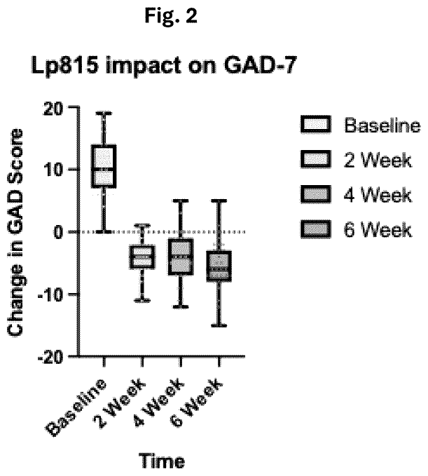

In some embodiments, the L. plantarum Lp815 strain causes a decrease in GAD-7 score (indicative of generalized anxiety) in a subject. In some embodiments, the L. plantarum Lp815 strain decreases the GAD-7 score by at least 10 points as compared to a baseline measurement. In some embodiments, the L. plantarum Lp815 strain decreases the GAD-7 score by at least 15 points as compared to a baseline measurement (see FIG. 2).

GABA can be readily identified by methods known in the art, including high-performance liquid chromatography (HPLC) and/or mass spectrometry.

The bacterial strain composition can be administered to a subject based on standard techniques known in the art for administration to the particular type of subject and in the environment in which the subject receives the bacterial strain composition. When administered to a human, the bacterial strain composition may be a liquid formulation or a solid formulation. The bacterial strain composition can be administered mucosally via oral administration, nasal administration, or rectal administration, for example, or administered parenterally, including but not limited to subcutaneous administration, transdermal administration, or any method that allows a bacterial strain of the invention to uptake glutamate.

In some embodiments of the invention, the method comprises administration of multiple doses of the bacterial composition to a subject. The method may comprise administration of 1, 2, 3, 4, 5, 6, 7, 8, 9, 10, 15, 20, 25, 30, 35, 40, or more effective doses of a bacterial strain composition. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to increase the production of isothiocyanate when compared to an appropriate control. It will also be appreciated that the effective amount or dosage of a bacterial strain composition may increase or decrease over the course of a particular treatment. Changes in dosage may result and become apparent from the results of diagnostic assays for detecting GABA levels known in the art and described herein.

As used herein, “treatment” or “treating” refers to therapeutic (e.g., curing, healing, alleviating, relieving, altering, remedying, ameliorating, improving, or affecting the condition or the symptoms of a subject) and preventative effects. The prophylactic administration (wherein a bacterial strain of the invention is administered in advance of symptoms) serves to prevent or attenuate any subsequent symptom. When provided therapeutically, the bacterial strain composition is provided at (or shortly after) the onset of a symptom. The therapeutic administration of the substance may serve to attenuate any actual symptom.

In one aspect, the bacterial compositions and formulations disclosed herein are applied topically (i.e., as a topical application) to a subject. In some embodiments, the composition applied topically comprises a L. plantarum Lp815 strain disclosed herein. In some embodiments, the composition applied topically comprises a supernatant of L. plantarum Lp815. In some embodiments, the composition applied topically comprises L. plantarum Lp815 in combination with a supernatant of L. plantarum Lp815. The compositions and formulations applied topically may further comprise glutamate, vitamin B6, and/or pyridoxal phosphate. Accordingly, in some embodiments, the composition or formulation applied topically comprises L. plantarum Lp815 with glutamate; L. plantarum Lp815 with vitamin B6; L. plantarum Lp815 with pyridoxal phosphate, or a combination thereof. In some embodiments, the composition or formulation applied topically comprises a supernatant of L. plantarum Lp815 with glutamate; a supernatant of L. plantarum Lp815 with vitamin B6; a supernatant of L. plantarum Lp815 with pyridoxal phosphate; or a combination thereof. In some embodiments, the composition or formulation applied topically comprises L. plantarum Lp815 and a supernatant of L. plantarum Lp815 with glutamate; L. plantarum Lp815 and a supernatant of L. plantarum Lp815 with vitamin B6; L. plantarum Lp815 and a supernatant of L. plantarum Lp815 with pyridoxal phosphate; or a combination thereof.

In some of the embodiments as described above, the Lactiplantibacillus plantarum Lp815 strain is NRRL Deposit No. B-68326.

### Methods of Treatment

Methods are provided herein for treating diseases or disorders caused by deficient levels of GABA. Without wishing to be bound by any particular theory, it is believed that a composition that comprises GABA, or comprises a bacterial strain composition that can produce GABA in vivo, can increase GABA levels such that one or more symptoms of the disease or disorder is alleviated. Accordingly, the compositions disclosed herein can be used to treat stress disorders, mood disorders, and sleep disorders.

In some embodiments, the mood disorder is major depression disorder, dysthymia, bipolar disorder, substance-induced mood disorders, social anxiety disorder, panic disorder, phobias, generalized anxiety disorder, and/or separation anxiety disorder.

In some embodiments, the sleep disorder is insomnia, narcolepsy, excessive daytime sleepiness, sleep phase disorders, circadian disorders, rapid eye movement behavioral disorders, and/or restless leg syndrome.

In some embodiments, the stress-related disorder is reactive attachment disorder, disinhibited social engagement disorder, post-traumatic stress disorder, acute stress disorder, and/or adjustment disorder.

The compositions disclosed herein can be used to increase levels of GABA in a subject suffering from one or more of the disorders disclosed herein. Such increases can include, for example, at least a 5%, 10%, 20%, 30%, 40%, 50%, 60%, 70%, 80%, 90%, 100%, 150%, 200% or greater increase in GABA levels when compared to an appropriate control, such as a subject that does not receive the composition. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in GABA levels when compared to an appropriate control, such as a subject that does not receive the composition. In some embodiments, the composition comprises supernatant produced by L. plantarum Lp815 and comprises GABA. In some embodiments the composition comprises supernatant produced by L. plantarum Lp815 and comprises both GABA and inactive cell fragments of L. plantarum Lp815. In some embodiments, the composition comprises a cell, spore, or forespore, or any combination thereof, of L. plantarum Lp815. In some embodiments, the composition comprises a cell, spore, or forespore, or any combination thereof, in conjunction with the supernatant produced thereof. Any method known in the art can be used to measure levels of GABA, including but not limited to liquid chromatography/mass spectrometry (LC-MS).

In one aspect, the compositions disclosed herein can affect the structure and/or function of a human subject. As used herein, “affecting the structure and/or function of a human subject” refers to the ability for a composition or formulation disclosed herein to modulate or impact one or more physiological functions of a human subject. Accordingly, the compositions and formulations disclosed herein may affect the structure and/or function of a human subject, wherein the structure and/or function is impacted by elevated levels of GABA. In some embodiments, an effective amount of the compositions and formulations disclosed herein may support a positive mood and/or help maintain a balanced emotional well-being. In some embodiments, an effective amount of the compositions and formulations disclosed herein may support cognitive function and/or help maintain mental clarity. In some embodiments, an effective amount of the compositions and formulations disclosed herein may help support the body's response to stress and/or aid in maintaining a calm and relaxed disposition. In some embodiments, an effective amount of the compositions and formulations disclosed herein may support restful sleep and/or help maintain a healthy sleep cycle. Impacts to the structure and/or function of a subject can be measured using methods known in the art.

In some of the embodiments as described above, the Lactiplantibacillus plantarum Lp815 strain is NRRL Deposit No. B-68326.

The following examples are offered by way of illustration and not by way of limitation.

### EXAMPLES

### Example 1: Synthesis of GABA In Vitro

The Lactiplantibacillus plantarum Lp815 strain was assessed for its ability to convert glutamate to GABA in vitro. Overnight, stationary phase cultures (16-18 h) of L. plantarum Lp815 were used to inoculate 3 mL of fresh media at an optical density of ~0.05 as measured at 600 nm by a spectrophotometer (OD₆₀₀). Multiple media types can be used, including rich media (e.g., MRS broth) or minimal media supplemented with the necessary components to support growth. The media can further be supplemented with L (+)-glutamic acid or a salt thereof and/or piperazine-N,N′-bis(2-ethanesulfonic acid) (PIPES). Cultures were grown under anerobic conditions at ~37° C. for 48 hours. The L. plantarum Lp815 strain resulted in an increase in GABA production (data not shown).

### Example 2: Strain Lp815 Induces GABA Production in Humans

The Lactiplantibacillus plantarum Lp815 strain was assessed for its ability to increase GABA levels in human participants. Eight healthy adults (5 female, 3 male) provided a urine sample at baseline (T=0). Urine was analyzed for GABA and 5-HIAA levels using the Neurotransmitter Basic Panel (available from Life Extension/National Diagnostics, Inc.). 5-HIAA is a metabolite of serotonin and indicative of serotonin levels in a subject. Participants with GABA levels in the high range at baseline (>4.7 mmol/g GABA) were excluded from the study. Participants proceeded to take the Lp815 probiotic at 1×10⁹ cfu/day for 4 weeks. Participants refrained from beginning any new treatments, using antibiotics, or taking any additional probiotic products.

After 4 weeks of supplementation, a second urine sample was collected and GABA levels measured. As shown in FIG. 1A, GABA levels increased by approximately 20% at week 4 relative to baseline. Additionally, levels of 5-HIAA were increased by approximately 10% at week 4 relative to baseline (FIG. 1B).

### Example 3: Strain Lp815 Decreases Anxiety in Humans

A double blind, randomized, placebo-controlled study was conducted to assess the impact of Lactiplantibacillus plantarum Lp815 on generalized anxiety scores. Participants with self-reported mild to moderate anxiety as measured by the GAD-7 questionnaire were enrolled in a six week trial. Participants with severe anxiety were excluded.

The GAD-7 questionnaire is a self-reported questionnaire that assesses anxiety levels based on 7 questions as measured over the last two weeks: 1) feeling nervous, anxious, or on edge, 2) not being able to stop or control worrying, 3) worrying too much about different things, 4) trouble relaxing, 5) being so restless that it is hard to sit still, 6) becoming easily annoyed or irritable, and 7) feeling afraid as if something awful might happen. For each question, a score of 0 (not at all), 1 (several days), 2 (more than half the days), or 3 (nearly every day) is assigned. The total score is a sum across the seven questions. A total score of 0-4 indicates minimal anxiety, 5-9 indicates mild anxiety, 10-14 indicates moderate anxiety, and 15-21 indicates severe anxiety.

Participants were divided into either placebo, 1×10⁹ CFU daily of Lp815 or 5×10⁹ CFU daily of Lp815. The GAD-7 questionnaire was administered at baseline, 2 weeks, 4 weeks and 6 weeks following supplementation. As shown in FIG. 2, supplementation with Lp815 resulted in a significant decrease of approximately 10-15 points in GAD-7 scores relative to baseline at weeks 2, 4, and 6.

We claim:

1. A bacterial strain composition comprising a bacterial cell of Lactiplantibacillus plantarum Lp815,
wherein the Lactiplantibacillus plantarum strain Lp815 is deposited as NRRL Deposit No. B-68326.

2. The bacterial strain composition of claim 1, wherein the Lactiplantibacillus plantarum Lp815 cell is present at about 10⁶ CFU/gram to about 10¹² CFU/gram or at about 10⁶ CFU/mL to about 10¹² CFU/mL.

3. The bacterial strain composition of claim 1, further comprising glutamate, pyridoxal phosphate, and/or vitamin B6.

4. The bacterial strain composition of claim 1, further comprising gamma-aminobutyric acid (GABA).

5. The bacterial strain composition of claim 1, wherein the bacterial strain composition is formulated as a capsule, gel, paste, tablet, powder, or liquid.

6. A pharmaceutical composition comprising a pharmaceutically acceptable carrier and the bacterial strain composition of claim 1.

7. A composition comprising a supernatant of Lactiplantibacillus plantarum Lp815, wherein the Lactiplantibacillus plantarum strain Lp815 is deposited as NRRL Deposit No. B-68326.

8. The composition of claim 7, wherein the supernatant further comprises cell fragments of Lactiplantibacillus plantarum Lp815.

9. The composition of claim 7, wherein the composition comprises gamma-aminobutyric acid (GABA).

10. The composition of claim 7, further comprising a bacterial cell of Lactiplantibacillus plantarum Lp815.

11. The composition of claim 7, wherein the composition is formulated as a capsule, gel, paste, tablet, powder, or liquid.

12. A pharmaceutical composition comprising a pharmaceutically acceptable carrier and the composition of claim 7.

13. A method of increasing gamma-aminobutyric acid (GABA) levels in a subject, the method comprising administering to the subject an effective amount of a composition comprising:
a) a bacterial cell of Lactiplantibacillus plantarum Lp815, an active variant of Lactiplantibacillus plantarum Lp815, or any combination thereof; and/or
b) a supernatant of Lactiplantibacillus plantarum Lp815, or an active variant thereof, and a pharmaceutically acceptable carrier,
wherein the Lactiplantibacillus plantarum strain Lp815 is deposited as NRRL Deposit No. B-68326,
wherein GABA levels are increased when compared to an appropriate control.

14. The method of claim 13, wherein the increase in GABA treats a disorder in the subject.

15. The method of claim 14, wherein the disorder is one or more disorders selected from the group consisting of: stress disorder, mood disorder, or sleep disorder.

16. The method of claim 13, wherein the composition further comprises one or more supplemental component selected from the group consisting of: glutamate, pyridoxal phosphate, and vitamin B6.

17. The method of claim 16, wherein the one or more supplemental component is simultaneously administered with the composition.

18. The method of claim 16, wherein the one or more supplemental component is sequentially administered with the composition, either before or after administration of the composition.

19. The method of claim 13, wherein the supernatant of Lactiplantibacillus plantarum Lp815 or active variant thereof comprises gamma-aminobutyric acid (GABA).

20. The method of claim 13, wherein the supernatant of Lactiplantibacillus plantarum Lp815 or active variant thereof further comprises cell fragments.

21. The method of claim 13, wherein the effective amount of the composition comprises about 10⁶ CFU/gram to about 10¹⁰ CFU/gram or at about 10⁶ CFU/mL to about 10¹⁰ CFU/mL of the Lactiplantibacillus plantarum Lp815 cell, active variant of Lactiplantibacillus plantarum Lp815 or any combination thereof.

22. The method of claim 13, wherein the effective amount of the composition comprises about 1×10⁹ to about 1×10¹⁰ CFU.

23. The method of claim 13, wherein the composition is administered orally to the subject.

24. The method of claim 13, wherein the composition is administered topically to the subject.

25. The method of claim 13, wherein the subject is a human subject.
